(19) United States
(12) Patent Application Publication
(10) Pub. No.: US 2016/0044882 A1
(43) Pub. Date: Feb. 18, 2016

(54) HYDROPONIC COMPOSITIONS AND APPLICATIONS THEREOF

(71) Applicant: THE UNIVERSITY OF NORTH CAROLINA AT GREENSBORO, Greensboro, NC (US)

(72) Inventor: Daniel Joseph Christian HERR, Chapel Hill, NC (US)

(21) Appl. No.: 14/782,117

(22) PCT Filed: Apr. 3, 2014
(86) PCT No.: PCT/US2014/032810

§ 371 Date: Oct. 2, 2015

Related U.S. Application Data
(60) Provisional application No. 61/808,031, filed on Apr. 3, 2013.

(51) Int. Cl. A01H 3/04; G01N 21/31; G01N 33/00

(52) U.S. Cl. CPC A01H 3/04; G01N 33/0098; G01N 21/31

(57) ABSTRACT

In one aspect, hydroponic compositions are described herein comprising an aqueous medium in contact with a plant, the aqueous medium comprising a functional additive for imparting desired architecture and/or properties to the plant structure. In some embodiments, the functional additive comprises one or more of nanoparticles, spectral additives, and pH indicators.

### CROSS REFERENCE TO RELATED APPLICATION

This application claims priority pursuant to 35 U.S.C. §119 to U.S. Provisional Patent Application Ser. No. 61/808,031, filed on Apr. 3, 2013, which is hereby incorporated by reference in its entirety.

### FIELD

The present invention relates to hydroponic compositions and, in particular, to hydroponic compositions operable to impart various functionalities to plant structures.

### BACKGROUND

The agriculture industry is responsible for fulfilling several essential human needs including food, clothing, shelter and, to some extent, energy. Crops and other plant types, therefore, have been the subject of intense study and research. For example, numerous plant species have been genetically modified to increase yield, resist drought and/or pestilence. Further, significant amounts of investment have been devoted to external compositions such as fertilizers and pesticides for facilitating plant growth and sustainability.

Conventional farming, however, is approaching yield limits with increased environmental exposure to soil treatments that adversely impact various ecosystems. Excess use of antibiotics, for example, raises the issue of antibiotic resistance in plants and humans. Further, environmental pollution resulting from fertilizers is becoming an increasingly alarming problem. Trace elements, including heavy metals deposited in fertilizer compositions, can accumulate over time yielding toxic levels in the soil. In view of these considerations, new techniques are required for imparting desirable properties and characteristics to plant species.

Similar trends are also evident in the textile industry wherein significant resources are dedicated to the development of advanced natural and synthetic fibers. Various sizings have been applied to fibers for improving tensile strength, softness, stain resistance, durability and breath-ability. A systemic problem to such fiber treatments is that they are necessarily surface treatments and, therefore, subject to erosion and wear. Erosion, wear and other degradative mechanisms can severely limit the utility of many fiber treatments necessitating introduction of alternative fiber architectures and treatments.

### SUMMARY

Compositions and methods are described herein which, in some embodiments, address the foregoing problems. For example, compositions and methods described herein are operable to impart desirable properties and characteristics to a variety of plant species in a controlled manner with little or no impact on neighboring ecosystems. Further, compositions and methods described herein can provide new fiber architectures demonstrating desirable mechanical and/or chemical properties which, in some embodiments, obviate the need for fiber surface treatments.

In one aspect, hydroponic compositions are described herein comprising an aqueous medium in contact with a plant, the aqueous medium comprising a functional additive for imparting desired architecture and/or mechanical properties to the plant structure. In some embodiments, for example, a hydroponic composition comprises an aqueous medium including nanoparticles and a plant in contact with the aqueous medium, wherein the nanoparticles are incorporated into the plant structure. In some cases, the nanoparticles have a plant binding functionality. Incorporation of the nanoparticles into plant structure can include binding to cell walls through the binding agent or passage through the cell wall and plasma membrane into the interior of the plant cells. Nanoparticles may also reside in extracellular space of the plant structure.

In another embodiment, a hydroponic composition comprises an aqueous medium including one or more spectral additives and a plant in contact with the aqueous medium, wherein the one or more spectral additives are incorporated into the plant structure altering the spectral properties of the plant. Spectral additives, such as various dyes, quantum dots and/or metal oxides, can be incorporated within plant cells or remain on the exterior of the cells. Further, spectral additives can also be provided a plant binding functionality for attaching along cell walls of the plant structure.

In a further embodiment, a hydroponic composition comprises an aqueous medium including one or more pH indicators and a plant in contact with the aqueous medium, wherein the one or more pH indicators are incorporated into a structure of the plant for monitoring pH local to the structure. Similar to above, pH indicators can be incorporated within plant cells or remain on the exterior of the cells. pH indicators can also be provided a plant binding functionality for attaching along cell walls of the plant structure.

In some embodiments, the aqueous medium of a hydroponic composition comprises a plurality of additives providing various functions. In such embodiments, multiple functionalities can be imparted to plants in a single hydroponic composition described herein. For example, the aqueous medium of a hydroponic composition can comprise a mixture of nanoparticles and spectral additives for altering the structural/mechanical and spectral properties of the plant. Further, pH indicators can also be added to the mixture for incorporation into the plant structure for monitoring pH local to the structure.

In another aspect, methods are provided for imparting desirable functional characteristics to various plant species. In some embodiments, a method described herein comprises providing an aqueous medium including nanoparticles and contacting a plant with the aqueous medium to provide a hydroponic composition and initiate uptake of the nanoparticles by the plant, wherein the nanoparticles are incorporated into the plant structure. In some cases, the nanoparticles have a plant binding functionality.

In another embodiment, a method comprises providing an aqueous medium including one or more spectral additives and contacting a plant with the aqueous medium to provide a hydroponic composition and initiate uptake of the one or more spectral additives by the plant, wherein the spectral additives are incorporated into the structure of the plant altering the spectral properties of the plant.

In a further embodiment, a method described herein comprises providing an aqueous medium including one or more pH indicators and contacting a plant with the aqueous medium to provide a hydroponic composition and initiate uptake of the one or more pH indicators by the plant, wherein the pH indicators are incorporated into a structure of the plant for monitoring pH local to the structure.

As described herein, the aqueous medium can comprise several additives for imparting various functionalities to the plant. For example, the aqueous medium can comprise any combination of nanoparticles, spectral additives and/or pH indicators for incorporation into plant structures.

The ability to impart desirable properties and characteristics to plant tissues through hydroponic compositions comprising functional additives marks a fundamental departure from prior plant developmental techniques relying on genetic modifications for inducing desired functional properties. These and other embodiments are described further in the detailed description which follows.

### BRIEF DESCRIPTION OF THE DRAWINGS

FIGS. 1 and 2 illustrate a method of providing a nanoparticle a plant binding functionality according to one embodiment described herein.

FIG. 3 illustrates an apparatus and a hydroponic composition according to one embodiment described herein.

FIG. 4 illustrates the Raman shift of carbon nanoparticles of a hydroponic composition according to one embodiment described herein.

FIG. 5 illustrates the Raman shift of carbon nanoparticles as a function of diameter.

FIGS. 6(a)-(b

) illustrate a tulip plant of a hydroponic composition wherein the aqueous medium contained a spectral additive responsive to near UV radiation according to one embodiment described herein.

FIGS. 7(a)-(b) illustrate a lettuce plant of a control composition.

FIGS. 8(a)-(b) illustrate a lettuce plant of a hydroponic composition wherein the aqueous medium contained a spectral additive responsive to near UV radiation according to one embodiment described herein.

FIGS. 9(a)-(b) illustrate a flax plant of a control composition.

FIGS. 10(a)-(b) illustrate a flax plant of a hydroponic composition wherein the aqueous medium contained a spectral additive responsive to near UV radiation according to one embodiment described herein.

FIGS. 11(a)-(b) illustrate a flax plant of a hydroponic composition wherein the aqueous medium contained a spectral additive responsive to near UV radiation according to one embodiment described herein.

FIGS. 12(a)-(b) illustrate a flax plant of a hydroponic composition wherein the aqueous medium contained a spectral additive responsive to near UV radiation according to one embodiment described herein.

FIGS. 13(a)-(b) illustrate a flax plant of a hydroponic composition wherein the aqueous medium contained a spectral additive responsive to near UV radiation according to one embodiment described herein.

FIGS. 14(a)-(c) illustrate a flax plant of a control composition and flax plants of hydroponic compositions wherein the aqueous medium contained a spectral additive responsive to near UV radiation according to one embodiment described herein.

FIG. 15 illustrates a Raman spectrum of a surfactant solution.

FIG. 16 illustrates a Raman spectrum of carbon nanoparticles disposed in the surfactant solution of FIG. 15.

FIGS. 17(a)-(c) illustrate the Raman shifts of a flax plant of a control composition at different locations along the stem fiber tissue of the plant.

FIGS. 18(a)-(e) illustrate the Raman shifts of a flax plant of a hydroponic composition according to one embodiment described herein at different locations along the stem fiber tissue of the plant.

FIG. 19 illustrates the Raman shift of the leaf tissue system of a flax plant of a hydroponic composition according to one embodiment described herein.

FIGS. 20(a)-(b) illustrate Raman images of nanoparticles observed in flax leaves of a hydroponic composition according to one embodiment described herein.

FIGS. 21(a)-(c) illustrate a control flax stem and a flax stem of a hydroponic composition according to one embodiment described herein wherein the aqueous medium contained a spectral additive responsive to near UV radiation.

### DETAILED DESCRIPTION

Embodiments described herein can be understood more readily by reference to the following detailed description, examples and drawings and their previous and following descriptions. Elements, apparatus and methods described herein, however, are not limited to the specific embodiments presented in the detailed description, examples and drawings. It should be recognized that these embodiments are merely illustrative of the principles of the present invention. Numerous modifications and adaptations will be readily apparent to those of skill in the art without departing from the spirit and scope of the invention.

### I. Hydroponic Compositions

In one aspect, hydroponic compositions are described herein comprising an aqueous medium in contact with a plant, the aqueous medium comprising a functional additive for imparting desired architecture and/or properties to the plant structure. In some embodiments, for example, a hydroponic composition comprises an aqueous medium including nanoparticles and a plant in contact with the aqueous medium, wherein the nanoparticles are incorporated into the plant structure. In some cases, the nanoparticles have a plant binding functionality. Incorporation of the nanoparticles into plant structure can include binding to cell walls through the binding agent or passage through the cell wall and plasma membrane into the interior of the plant cells. Nanoparticles may also reside in extracellular space of the plant structure.

Normal plant transport processes can vary the distribution of nanoparticles within plant cells and tissue, including in a manner that provides a gradient of nanoparticle sizes within the plant. For example, in some instances, a plant can comprise a gradient of nanoparticles, wherein the average size of the nanoparticles in at least one dimension decreases toward the growing tips or extremities of the plant. Thus, in some embodiments, hydroponic compositions described herein can be used to size select a population of nanoparticles.

Further, in some embodiments, plant transport processes can be exploited for targeted or localized delivery of nanoparticles within the plant structure. For example, nanoparticles can be localized to fibrous tissue of the plant structure binding to cell walls of the fibrous tissue. In some embodiments, nanoparticles are provided to areas of meristematic tissue including apical meristems and lateral meristems. In other instances, nanoparticles are provided to stems and/or leaves of a plant. Nanoparticles can also be provided to the growing tips or growing regions of a plant, including the growing tips of stems and/or leaves of the plant.

It is also possible for nanoparticles to be localized within the cells of a plant structure. For example, in some cases, nanoparticles can be localized in the cytoplasm of a cell or in an organelle of a cell. An organelle can be a mitochondria or a plastid such as a chloroplast. Nanoparticles may also be localized in other organelles.

Binding of nanoparticles to cell walls in one or more locations of the plant structure can enhance mechanical properties of fibers manufactured from the plant-nanoparticle composite. As described further herein, nanoparticles can enhance the tensile strength and/or flexural strength of various fibers including surface fibers of cotton and soft/bast fibers of flax, hemp, jute and/or ramie.

Further, nanoparticles can also enhance spectral properties of the foregoing fibers. For example, nanoparticles reflecting or absorbing various regions of the electromagnetic spectrum can be bound or otherwise incorporated into cells or tissue forming fibrous regions of the plant structure. In being bound or otherwise incorporated into the structure of the fibrous tissue, nanoparticles of desired functionality demonstrate an increased resistance to erosion and wear. Moreover, fibers of plants of hydroponic compositions described herein, in some embodiments, can exhibit enhanced mechanical and/or spectral properties even after the fibers are harvested and used to form fiber products such as textiles, including clothing. This is a significant advantage over prior surface treatments such as sizings aimed at enhancing fiber mechanical and/or spectral properties.

Turning now to specific components, any nanoparticles not inconsistent with the objectives of the present invention can be used in the aqueous medium for uptake by and incorporating in the plant structure of the hydroponic composition. In some embodiments, nanoparticles can comprise carbon nanoparticles. Suitable carbon nanoparticles can include fullerenes, single-walled carbon nanotubes (SWNT), multi-walled carbon nanotubes (MWNT), graphene or mixtures thereof.

Moreover, nanoparticles can also comprise quantum dots. Quantum dots, in some embodiments, are formed of III/V or II/VI semiconductor materials. For example, quantum dots can comprise indium arsenide (InAs), zinc selenide (ZnSe), zinc sulfide (ZnS), indium phosphide (InP), indium gallium arsenide (InGaAs), cadmium sulfide (CdS), cadmium selenide (CdSe) and/or cadmium telluride (CdTe). III/V and II/VI semiconductor materials forming quantum dots can be intrinsic or doped. Suitable dopants include various metal ions such as manganese, copper, erbium, ytterbium, dysprosium, holmium or combinations thereof.

Further, nanoparticles also comprise various metal oxides. In some embodiments, for example, nanoparticles comprise a transition metal oxide, including oxides of Group IVB metals. For example, nanoparticles of titania (TiO₂) or zinc oxide (ZnO) can be employed in hydroponic compositions described herein. Nanoparticles of silica (SiO₂) may also be used. In other cases, nanoparticles comprise a lanthanide or rare earth oxide such as ceria (CeO₂) or an oxide of dysprosium (Dy), holmium (Ho), gadolinium (Gd), terbium (Tb), erbium (Er), europium (Eu) or a combination thereof, such as DyₓEu_{y}O₃.

An aqueous medium can also comprise a combination of differing types of nanoparticles described hereinabove. For example, in some embodiments, nanoparticles of an aqueous medium comprise carbon nanoparticles and quantum dots; carbon nanoparticles and metal oxide nanoparticles; or metal oxide nanoparticles and quantum dots.

Nanoparticle dimensions can be varied to direct or facilitate placement of the nanoparticles in desired regions of the plant structure. In some embodiments, for example, nanoparticles have dimensions less than 10 nm or less than 5 nm for passage of the nanoparticles through cell wall and plasma membrane structures and into intracellular spaces. Alternatively, nanoparticles can have dimensions sufficiently large to inhibit or preclude intracellular placement.

Moreover, in some cases, nanoparticles have a size distribution in at least one dimension that facilitates size selection of the nanoparticles by the plant structure in a manner described above. For example, in some embodiments, nanoparticles of a hydroponic composition can comprise a first subpopulation having a first size distribution and a second subpopulation having a second size distribution, wherein the first and second size distributions differ. The first size distribution (e.g., a size distribution having a smaller average nanoparticle size in at least one dimension) may be selected for placement of the nanoparticles in a first region of the plant structure (e.g., in the growing tips of leaves of the plant), and the second size distribution (e.g., a size distribution having a larger average nanoparticle size in at least one dimension) may be selected for placement of the nanoparticles in a second region of the plant structure (e.g., in a stem of the plant).

It is also possible to direct or facilitate placement of nanoparticles in a desired region of a plant structure based on the electric charge or surface functionalization of the nanoparticles. For example, in some cases, nanoparticles of hydroponic compositions described herein have a positive or negative surface charge to facilitate passage of the nanoparticles across a membrane such as a cellular or subcellular membrane. In other instances, nanoparticles have a zwitterionic charge to facilitate localization of the nanoparticles in extracellular spaces of a plant structure. Surface charges of nanoparticles, in some embodiments, can be provided by a chemical species bound or associated with the surface of the nanoparticles. For instance, in some cases, a negative surface charge can be provided by a chemical species comprising a first moiety that associates with the surface of nanoparticles and a second moiety, such as a carboxylate moiety, that contacts the aqueous environment of the nanoparticles and is negatively charged at the pH of the aqueous environment. In other instances, the surface of nanoparticles can be functionalized with a targeting species such as an antibody that preferentially binds with a target location in a plant structure, including in a highly specific and selective manner.

For example, in some embodiments, nanoparticles of hydroponic compositions described herein can have associated plant binding functionalities. Plant binding functionalities can link or couple nanoparticles to one or more plant structures. For example, a plant binding functionality can couple the associated nanoparticle to a cell wall. Plant binding functionalities can comprise primary plant metabolites, secondary plant metabolites or combinations thereof. For example, a plant binding functionality of a nanoparticle can comprise one or more sugars, amino acids, proteins, nucleic acids or lipids. Sugars suitable for a plant binding functionality include glucose, sucrose, fructose, maltose, manose and lactose. Further, trace elements needed for plant growth can also serve as plant binding functionalities. Ions of copper, zinc, manganese, iron, molybdenum and boron can function as plant binding functionalities.

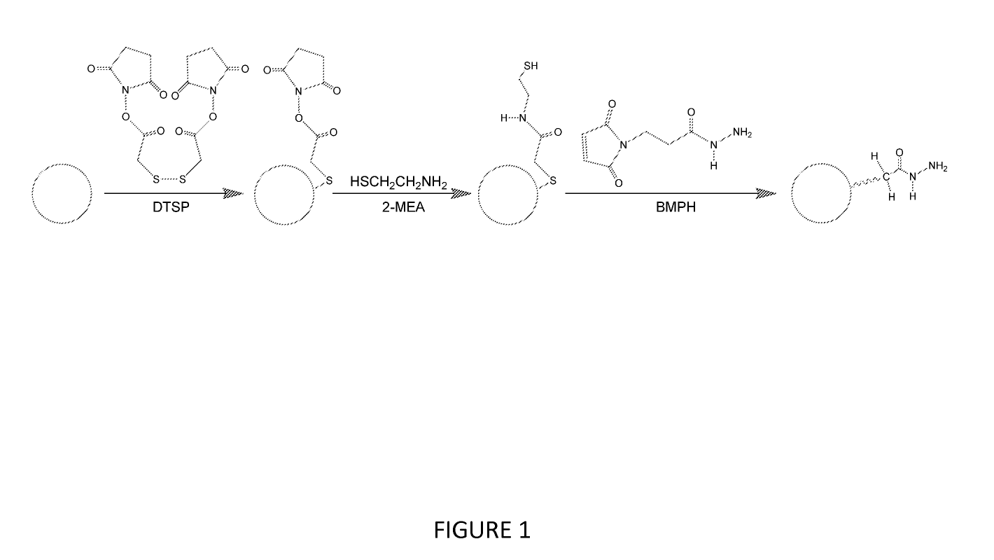

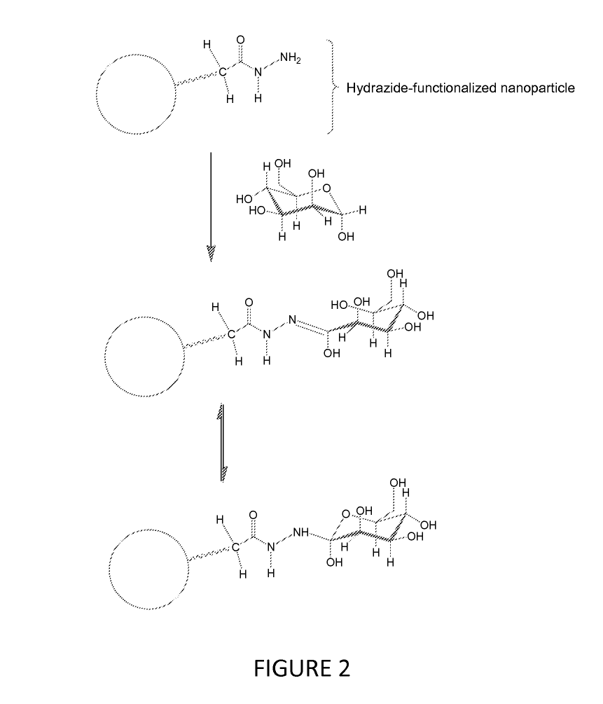

FIGS. 1 and 2 illustrate a method of providing a nanoparticle with a plant binding functionality according to one embodiment described herein. As illustrated in FIG. 1, the nanoparticle is initially functionalized with a hydrazide structure followed by reaction with the sugar of FIG. 2 to complete the metabolite plant binding functionality. In FIG. 1, coupling is carried out using a succinimidyl alkanoate such as 3,3′-dithiodipropionic acid di(N-hydroxysuccinimide ester), a mercaptoalkylamine such as 2-mercaptoethanolamine (2-MEA), and a hydrazide such as N-β-maleimidopropionic acid hydrazide (BMPH). However, other coupling schemes may also be used.

Nanoparticles can be present in the aqueous medium of the hydroponic composition in any amount not inconsistent with the objectives of the present invention. Nanoparticles can be present in an amount sufficient to effectuate the desired uptake and incorporation of the nanoparticles into the plant structure. Further, nanoparticles can be present in the aqueous medium for the entire development period of the plant. Alternatively, nanoparticles can be present in the aqueous medium at various intervals of the plant development period. Additionally, nanoparticle concentration can remain static or vary over growth and/or development periods of the plant. In some embodiments, nanoparticles are present in the aqueous medium of the hydroponic composition in a concentration up to about 100 millimolar (mM), up to about 10 mM, up to about 5 mM, or up to about 1 mM. In some cases, nanoparticles are present in the aqueous medium in a concentration between about 0.01 mM and about 10 mM, between about 0.05 mM and about 5 mM, or between about 0.1 mM and about 1 mM.

In some embodiments, other functional additives can be provided in the aqueous medium having the ability to impact structural, mechanical and/or textural properties of the plant structure and fibers resulting therefrom. For example, lanolin and quaternary ammonium salts, such as dipalmitoylethyl hydroxyethylmonium methosulfate and dehydrogenated tallow dimethyl ammonium chloride, can be added to the aqueous medium for uptake and incorporation into the plant structure. Additionally, scented agents including limonene and a-terpineol can be added to the aqueous medium as well as skin softeners such as natural oils and glycerin derivatives. The foregoing functional additives can replace nanoparticles in the aqueous medium or be present in addition to the nanoparticles. Moreover, the foregoing functional additives can be present in the aqueous medium in any amount not inconsistent with the objectives of the present invention. In some cases, for instance, one or more functional additives are present in the aqueous medium of the hydroponic composition in a concentration up to about 100 mM, up to about 10 mM, up to about 5 mM, or up to about 1 mM. In some cases, one or more functional additives are present in the aqueous medium in a concentration between about 0.01 mM and about 10 mM, between about 0.05 mM and about 5 mM, or between about 0.1 mM and about 1 mM.

The aqueous medium of hydroponic compositions described herein can be contained in a vessel of desired dimensions, wherein the plant is arranged to at least have the root structure in contact with the aqueous medium. In some embodiments, the plant is a cut structure wherein the roots are not present and the aqueous medium is in direct contact with the xylem and/or other vasculature of the plant. In some embodiments, use of cut structures can facilitate nanoparticle transport to the desired location in the plant structure by avoiding unnecessary complications that can occur with nanoparticle interaction with root structures.

Alternatively, the aqueous media is not contained in a vessel for contact with the plant. In such embodiments, the aqueous medium can be misted or provided as an aerosol for contact with the root structure or vasculature of the plant. This arrangement is sometimes referenced as an aeroponic composition.

In another embodiment described herein, a hydroponic composition comprises an aqueous medium including one or more spectral additives and a plant in contact with the aqueous medium, wherein the one or more spectral additives are incorporated into the structure of the plant altering the spectral properties of the plant. In some embodiments, for example, spectral additives are luminescent species such as fluorescent or phosphorescent species having absorption spectra non-overlapping with chlorophylls a and b. Moreover, the fluorescent and/or phosphorescent species can have emission spectra overlapping with the absorption spectra of chlorophylls a and b. In such embodiments, fluorescent and/or phosphorescent additives can expand the usable range of the electromagnetic spectrum for photosynthetic processes by the plant. Fluorescent and/or phosphorescent additives can downconvert ultraviolet (UV) radiation, near-UV radiation, or visible radiation for absorption by chlorophylls a and b. Fluorescent and/or phosphorescent additives may also upconvert infrared (IR) radiation for absorption by chlorophylls a and b. Further, fluorescent and/or phosphorescent additives may absorb radiation in the 500-600 nm range for re-emission at wavelengths suitable for absorption by chlorophylls a and b.

Alternatively, spectral additives may have an absorption profile substantially overlapping with that of chlorophylls a and b. Additionally, spectral additives may reflect light in the visible region of the electromagnetic spectrum. In such embodiments, the spectral additives can compete with photosynthetic processes and serve as herbicidal compositions.

Spectral additives can comprise a variety of materials including quantum dots or other nanoparticles, laser dyes, anti-Stokes materials, anti-counterfeiting dye, carbon nanoparticles or mixtures thereof. In some embodiments, a spectral additive comprises APC-Cy7 conjugates, aminocoumarin, fluorescein, xanthene, cyanine, naphthalene, coumarin, coumarin derivatives, oxadiazole, pyrene, oxazine, acridene, arylmethine, tretrapyrrole or mixtures thereof. Suitable spectral additives, in some embodiments, are commercially available under the trade designations CF dye, BODIPY, Alexa Fluor, DyLight Fluor, Atto and Tracy, FluoProbes, DY and MegaStokes, Sulfo Cy dyes, Setau and Square Dyes, Quasar and Cal Fluor dyes, SureLight Dyes, APC, APCXL, RPE and BPE. Further, quantum dot spectral additives can comprise any of the III/V and II/V constructions described hereinabove. Similarly, carbon nanoparticle spectral additives can comprise any of the carbon nanoparticles described hereinabove. Moreover, metal oxide nanoparticles, such as TiO₂, can be used as light blocking or reflective spectral modifiers. Other nanoparticles can include inorganic or organometallic nanotubes or nanosheets, such as molybdenum sulfide nanotubes or nanosheets. Other materials may also be used.

Spectral additives can be located in various structures of the plant of the hydroponic composition. Spectral additives, for example, can be located within and/or exterior to fibrous tissue cells, including in a manner described hereinabove for nanoparticles of a hydroponic composition. In such embodiments, the spectral additives can provide desirable spectral properties to fibers constructed from the plant of the hydroponic composition. Spectral additives may also be located in leaves of the plant for enhancement or inhibition of photosynthetic processes.

In some embodiments, spectral additives described herein are modified with a plant binding functionality. Spectral additives, for example, can be modified with any plant binding functionality described above for nanoparticle compositions, including primary and secondary plant metabolites. Modification with a plant binding functionality can increase interaction of the spectral additive with the plant structure to preclude or inhibit leaching of the spectral additive.

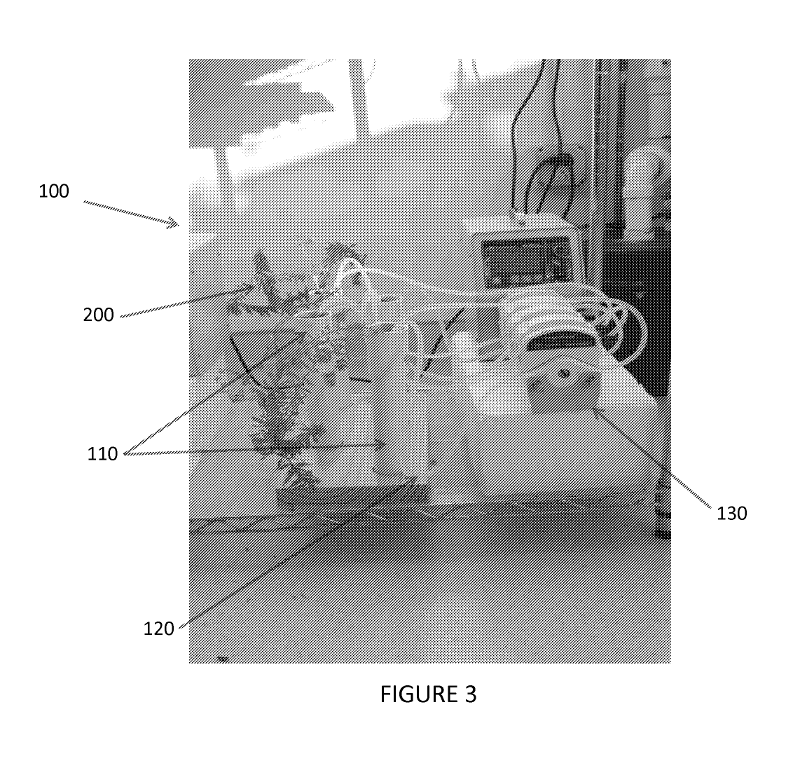

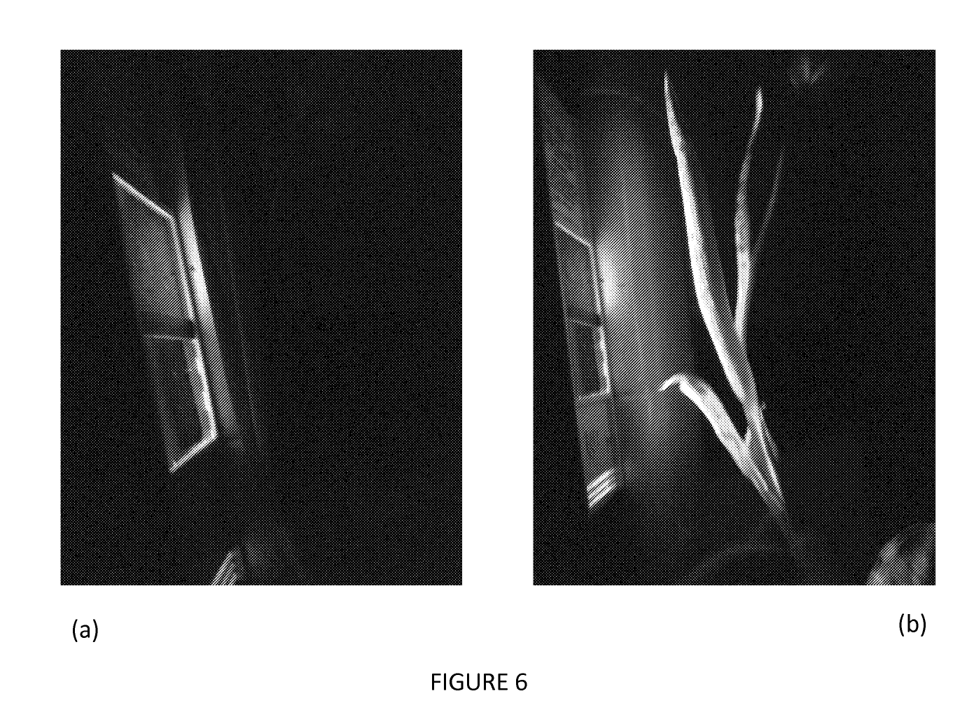

FIG. 6 illustrates a tulip plant of a hydroponic composition wherein the aqueous medium contained a spectral additive responsive to near UV radiation. As illustrated in FIG. 3, the tulip plant composite (b) comprising the UV spectral additive demonstrated an energy harvesting response to incident UV radiation whereas the untreated control tulip plant (a) was unresponsive to the incident UV radiation.

Spectral additives can be present in the aqueous medium of the hydroponic composition in any amount not inconsistent with the objectives of the present invention. A spectral additive can be present in an amount sufficient to effectuate the desired uptake and incorporation of the nanoparticles into the plant structure. Further, spectral additives can be present in the aqueous medium for the entire development period of the plant. Alternatively, spectral additives can be present in the aqueous medium at various intervals of the plant development period. Additionally, spectral additive concentration can remain static or vary over growth and/or development periods of the plant. In some embodiments, spectral additives are present in the aqueous medium of the hydroponic composition in a concentration up to about 100 mM, up to about 10 mM, up to about 5 mM, or up to about 1 mM. In some cases, spectral additives are present in the aqueous medium in a concentration between about 0.01 mM and about 10 mM, between about 0.05 mM and about 5 mM, or between about 0.1 mM and about 1 mM.

In another embodiment described herein, a hydroponic composition comprises an aqueous medium comprising one or more pH indicators and a plant in contact with the aqueous medium, wherein the one or more pH indicators are incorporated into a structure of the plant for monitoring pH local to the plant structure. Similar to above, pH indicators can be incorporated within plant cells or remain on the exterior of the cells. pH indicators can also be provided a plant binding functionality for attaching along cell walls of the plant structure or for localization in a different region of the plant.

Suitable pH indicators can comprise chemical species demonstrating a color change in response to pH local to the plant structure. Moreover, suitable pH indicators can also comprise chemical species produced in response to a pH change in the plant structure and, therefore, are not required to demonstrate a color change. In some embodiments, pH indicator(s) are selected from the group consisting of phenolphthalein, derivatives of Indo-1, Fluo-3, Fluo-4, oxidized-2′7′-dichlorodihydrofluorescein and oxidized dihydrorhodamine 123.

Hydroponic feed stream concentrations of pH indicators can be dependent upon plant uptake rates and localization concentration in each plant tissue, which is tuned for detectability and avoidance of bleaching. In some embodiments, pH indicators are present in the aqueous medium of the hydroponic composition in a concentration up to about 100 mM, up to about 10 mM, up to about 5 mM, or up to about 1 mM. In some cases, pH indicators are present in the aqueous medium in a concentration between about 0.01 mM and about 10 mM, between about 0.05 mM and about 5 mM, or between about 0.1 mM and about 1 mM.

As described herein, the aqueous medium, in some embodiments, can comprise several additives for imparting various functionalities to the plant. For example, the aqueous medium can comprise any combination of nanoparticles, spectral additives and/or pH indicators for incorporation into plant structures. Further, single additives can be engineered to provide multiple functionalities to the plant.

Hydroponic compositions described herein contemplate the use of a variety of plants for modification with various functional additives. As described herein, plants for use in hydroponic compositions described herein include plants of full structure comprising a root system as well as portions of plants, such as cut plants, stems and/or leaves. Thus, hydroponic compositions described herein can be used in in vivo or ex vivo contexts. Plants yielding surface fibers, soft/bast fibers and hard/structural fibers are particularly useful for textile applications. In some embodiments, for example, plants listed in Table I can be modified according to hydroponic compositions described herein.

TABLE I
Plants of Hydroponic Compositions for Textile Applications

| Surface Fibers | Soft/Bast Fibers | Hard/Structural Fibers |
|---|---|---|
| Cotton (Malvaceae/Gossypium spp.) | Flax (Linaceae/Linum | Abaca/Manila Hemp (Musaceae/ |
| Sea Island/Egyptian cotton | usitatissimum) | Musa textilis) |
| (Malvaceae/Gossypium barbadense) | Hemp/Marijuana | Agaves (Agavaceae Asparagaceae/ |
| Upland cotton/bulak/gapas/algodon | (Cannabaceae/Cannabis sativa) | Agave spp.) |
| (Malvaceae/Gossypium hirsutum) | Jute (Malvaceae/Corchorus | Henequen or Mexican Sisal |
| Tree cotton (Malvaceae/Gossypium | capsularis) | (Agavaceae Asparagaceae/Agave |
| arboreum), and Levant cotton | Jute/saluyot/tagabang (Malvaceae/ | fourcroydes) |
| (Malvaceae/Gossypium herbaceum) | Corchorus olitorius) | Sisal/Century plant (Agavaceae |
| | Ramie (Urticaceae/Boehmeria | Asparagaceae/Agave sisalana) |
| | nivea) | Maguey/Manila maguey/Cantala |
| | Sunn hemp (Leuminosae Fabaceae/ | (Agavaceae Asparagaceae/Agave |
| | Crotolaria juncea) | cantala) |
| | Kenaf (Malvaceae/Hibiscus | Mauritius Hemp/False agave/green |
| | cannabinus) | aloe/giant cabuya (Agavaceae |
| | China Jute/Indian Mallow | Asparagaceae/Furcraea gigantea, |
| | (Malvaceae/Abutilon theophrasti) | F. foetida |
| | Roselle/Rama (Malvaceae/Hibiscus | |
| | sabdariffa) | |
| | Aramina/Cadillo/Calut-calutan | |
| | (Malvaceae/Urena lobata) | |

Other plants for modification with hydroponic compositions described herein include Furcraea acrophylla, F. cabuya, F. hexapetales, New Zealand Flax/New Zealand Hemp (Hemerocallidaceae Xanthorrhoeaceae/Phormeum tenax), Bowstring Hemp (Ruscaceae Asparagaceae/Sansevieria spp., e.g. S. thyrsiflora, S. roxburghiana, S. zelanica, S. longifolia), Coconut (Palmae Arecaceae/Cocos nucifera), Pineapple (Bromeliaceae/Ananas comosus), Floja (Bromeliaceae/Aechmea magdalenae), and Caroá (Floja (Bromeliaceae/Floja (Bromeliaceae/Aechmea magdalenae).

### II. Methods of Imparting Plant Functionalities

In another aspect, methods are provided for imparting desirable functional characteristics to various plant species. In some embodiments, a method described herein comprises providing an aqueous medium including nanoparticles and contacting a plant with the aqueous medium to provide a hydroponic composition and initiate uptake of the nanoparticles by the plant, wherein the nanoparticles are incorporated into the plant structure. In some cases, the nanoparticles have a plant binding functionality

In another embodiment, a method comprises providing an aqueous medium including one or more spectral additives and contacting a plant with the aqueous medium to provide a hydroponic composition and initiate uptake of the one or more spectral additives by the plant, wherein the spectral additives are incorporated into the structure of the plant, thereby altering the spectral properties of the plant or a portion of the plant.

In a further embodiment, a method described herein comprises providing an aqueous medium including one or more pH indicators and contacting a plant with the aqueous medium to provide a hydroponic composition and initiate uptake of the one or more pH indicators by the plant, wherein the pH indicators are incorporated into a structure of the plant for monitoring pH local to the structure.

Some embodiments described herein are further illustrated in the following non-limiting examples.

### EXAMPLE 1

### Hydroponic Compositions

Hydroponic compositions according to some embodiments described herein were generally prepared as follows using an apparatus similar to that illustrated in FIG. 3. The apparatus (100) of FIG. 3 comprises a plurality of test vessels (110), where each vessel (110) includes an intake tube (120) connected to a peristaltic pump (130). The intake tube (120) is connected on the other end to an output needle (not shown). The output needle can be used to direct the flow of a fluid provided by the intake tube (120) to a desired location, such as to a region near the root system of a plant (200) disposed in a test vessel (110). Such apparatus permitted various plants to be contacted with various aqueous solutions described herein at tunable flow rates. The peristaltic pumps delivered the aqueous solutions to each plant root system at a rate of 10-70 mL per minute through the intake tubes. In addition, each plant was exposed to approximately 18 hours of illumination each day. The illumination source comprised fluorescent or high intensity discharge lamps.

Aqueous solutions were prepared as follows. First, 4 gallons of tap water (pH approximately 7.4) was allowed to stand for 1 hour. The water was then titrated to pH=6.0 with an acid, such as phosphoric acid, while stirring. The water having a pH=6.0 was then used to prepare solutions comprising nanoparticles, spectral additives, pH indicators, and/or other functional additives described herein.

Specifically, to prepare and deliver aqueous solutions comprising a dye, 0.25 g of dye was dissolved in 100 mL of the pH=6.0 water prepared above. Typical dyes included D-282

(CAS number 41085-99-8) and OB-M1 (CAS number 27344-41-8). The dye solutions were then poured into the test vessels of an apparatus such as that described above. Next, a plant or a portion of a plant, such as flax in rock wool, was inserted into the top of a test vessel of the apparatus, with the intake tube already extended to the bottom of the test vessel. The output needle of the tube was inserted into the rock wool, near the top of the plant's root system. The pump was then turned on and the pump start time was recorded.

To prepare and deliver aqueous solutions comprising nanoparticles, the same procedure above for dyes was used, except the amounts of nanoparticles used were as follows: (a) 0.5 mM alumina nanoparticles (average size approximately 30 nm), stabilized in solution with 0.5% F-68 surfactant; (b) 0.5 mM silica nanoparticles (average size approximately 12 nm), stabilized in solution with 0.5% F-68 surfactant; (c) 0.5 mM ZnO nanoparticles (average size <100 nm), stabilized in solution with 0.5% F-68 surfactant; (d) 1 mL of saturated single walled carbon nanotubes (SWNT) (average diameter approximately 0.78 nm, average length approximately 1.5 μm), dispersed with 0.4% sodium dodecyl sulfate (SDS) and 1.6% sodium cholate (SC), diluted to 100 mL with H₂O (pH=6.0); and (e) 1 mL of graphene solution (Angstrom Materials: N002-PS: Lot s2011912; average sheet length <10 μm; 0.5% solid content) was diluted to 100 mL with H₂O (pH=6.0). Properties of the SWNTs are provided in Table II below.

TABLE II
SWNT Properties

| Parameter | Value | Method |
|---|---|---|
| Appearance and Form | Freeze Dried Black | |
| | powder | |
| Bulk Density (Tapped) | 0.094 g cm⁻³ | |
| Moisture Content | <5% | By weight (TGA) |
| Carbon Content | >95% | TGA |
| Residual Mass | ≦5% | TGA |
| Semiconducting Tube Content | ≧95 | OA |
| (6,5) Content | >40 | NIRF |
| Average Diameter (nm) | 0.78 | NIRF |
| Raman Quality parameter (Q) | ≧0.97 | Raman |
| Median Tube Length (μm) | 1.5 | AFM |
| Specific Surface Area (m²/g) | 790 | BET |

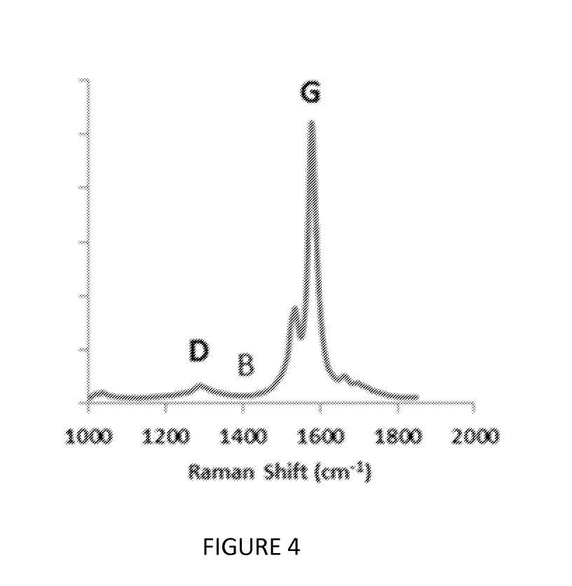

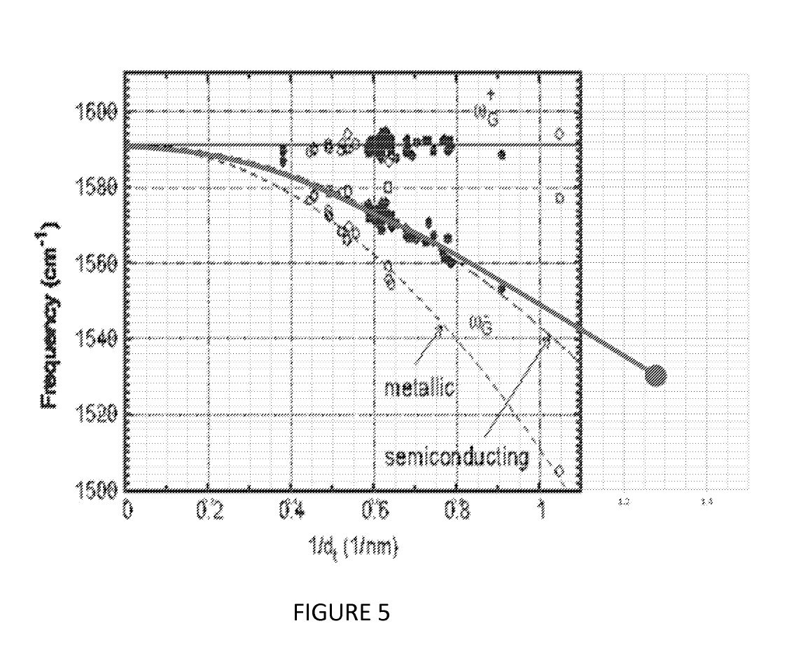

In addition, FIG. 4 illustrates Raman shifts of the SWNTs. More generally, FIG. 5 illustrates Raman shift trends of SWNTs as a function of SWNT diameter.

To characterize the plants of the hydroponic compositions above, the following methods were used. Visual plant inspection was performed under ambient lab lighting conditions. UV plant inspection, to examine macroscopic fluorescent plant tissue properties, was performed using a UV light that emits at 254 nm and 365 nm. Confocal microscopy was also performed, specifically, 3D scans through approximately 100 μm of sample plant tissues, exposed to 405 nm, 488 nm, and 561 nm laser irradiation, were gathered and analyzed. Raman spectra of sample plant tissues, exposed to NIR (785 nm) laser irradiation, were gathered and analyzed. A typical illumination spot size was approximately 1-5 μm. Inductively Coupled Plasma (ICP) analysis was used to determine the concentrations of metals in the plant tissue samples that underwent nanoparticle uptake experiments. Typically, plant tissue sample preparation began by drying the tissues, in an open ended cuvette, in an 85° C. oven for 12-24 hours. The cuvettes, containing the dried tissue, are then filled with liquid nitrogen to disrupt cell membranes. Once the liquid nitrogen evaporated, the samples were digested with nitric acid, overnight, in an oven. The digested samples were filtered and introduced into the ICP and atomic emissions at element-specific wavelengths were measured.

### EXAMPLE 2

### Hydroponic Composition

A hydroponic composition according to one embodiment described herein was prepared according to Example 1. Specifically, a cut tulip was contacted with an aqueous solution comprising a yellow fluorescent dye. As illustrated in FIG. 6(b), the dye-containing tulip exhibited fluorescence under UV irradiation. In contrast, as illustrated in FIG. 6(a), a control cut tulip that was not contacted with the aqueous solution containing the dye did not exhibit fluorescence.

### EXAMPLE 3

### Hydroponic Composition

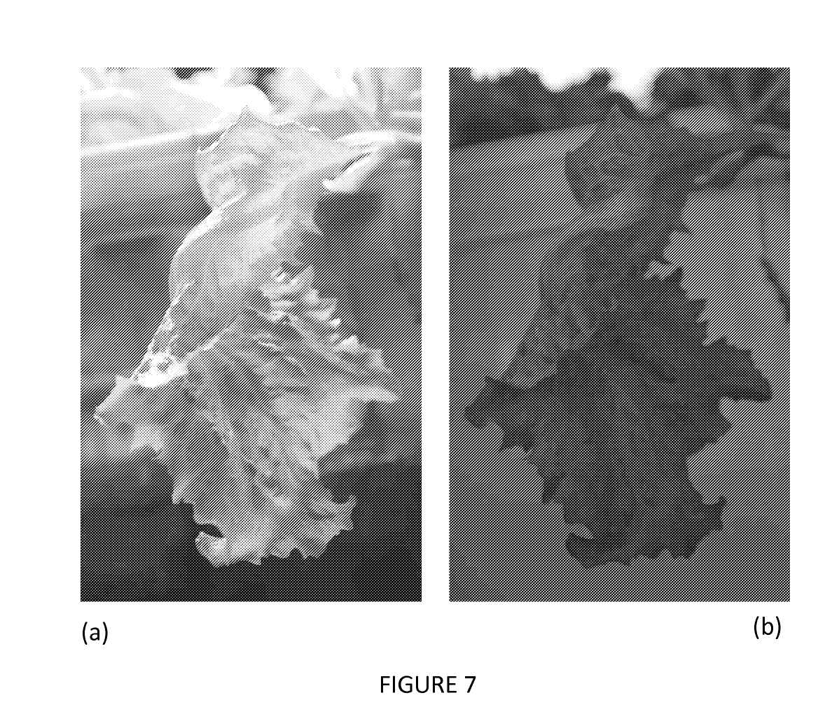

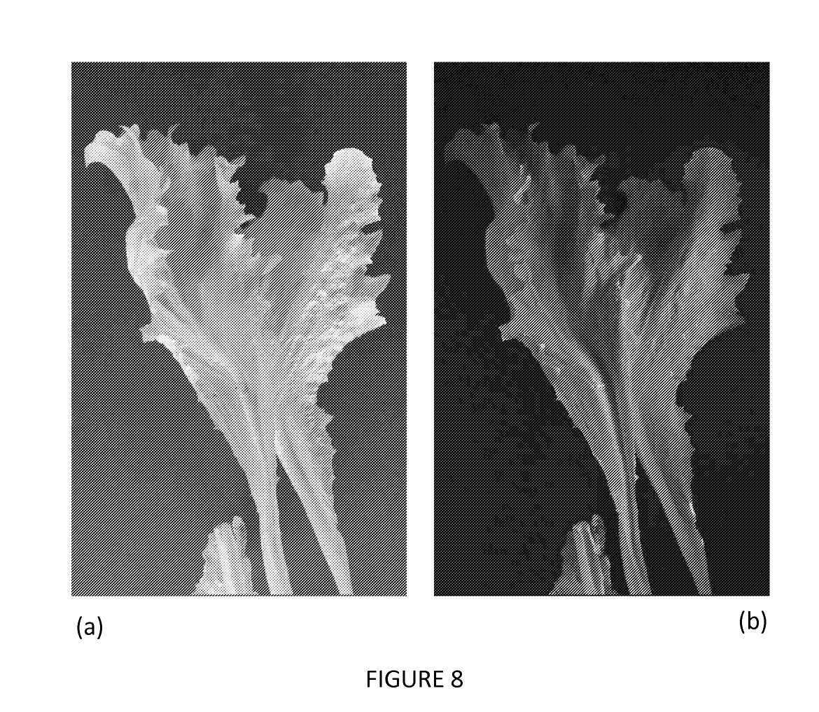

A hydroponic composition according to one embodiment described herein was prepared according to Example 1. Specifically, a lettuce was contacted with an aqueous solution comprising D-282 dye. As illustrated in FIG. 8, the dye-containing lettuce exhibited fluorescence under UV irradiation. In contrast, as illustrated in FIG. 7, a control lettuce that was not contacted with the aqueous solution containing the dye did not exhibit fluorescence. FIGS. 7(a) and 8(a) are photographs of the lettuces when exposed to ambient visible light. FIGS. 7(b) and 8(b) are photographs of the lettuces when exposed to UV light.

### EXAMPLE 4

### Hydroponic Compositions

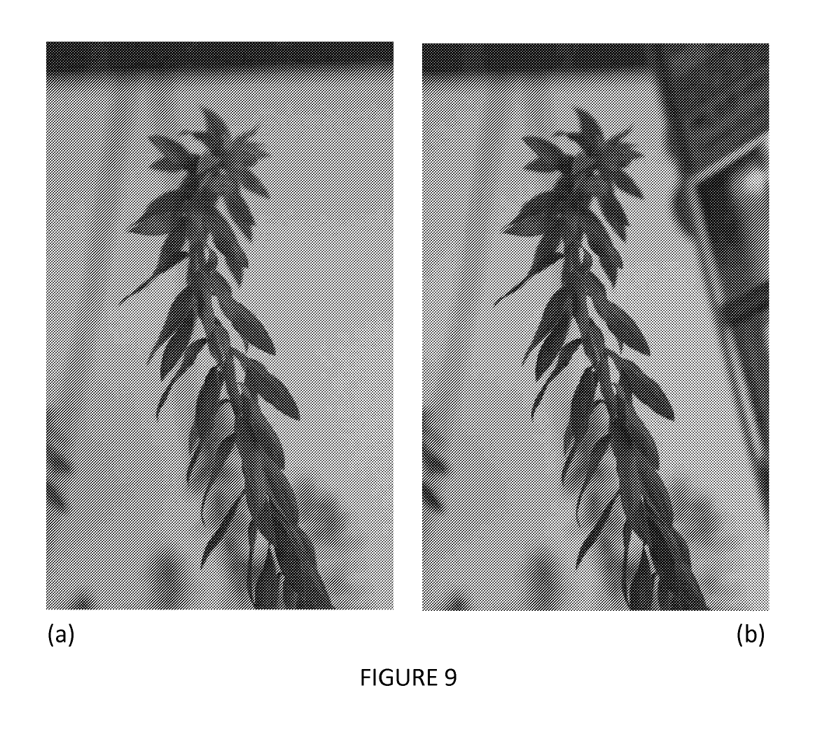

Hydroponic compositions according to some embodiments described herein were prepared according to Example 1. Specifically, flax plants or parts of flax plants were contacted with aqueous solutions comprising various nanoparticles and spectral additives described herein. Some results are illustrated in FIGS. 9-23.

FIG. 9 illustrates a control flax plant that was not part of a hydroponic composition described herein. FIG. 9(a) is a photograph of the flax plant when exposed to ambient visible light. FIG. 9(b) is a photograph of the flax plant when exposed to UV light.

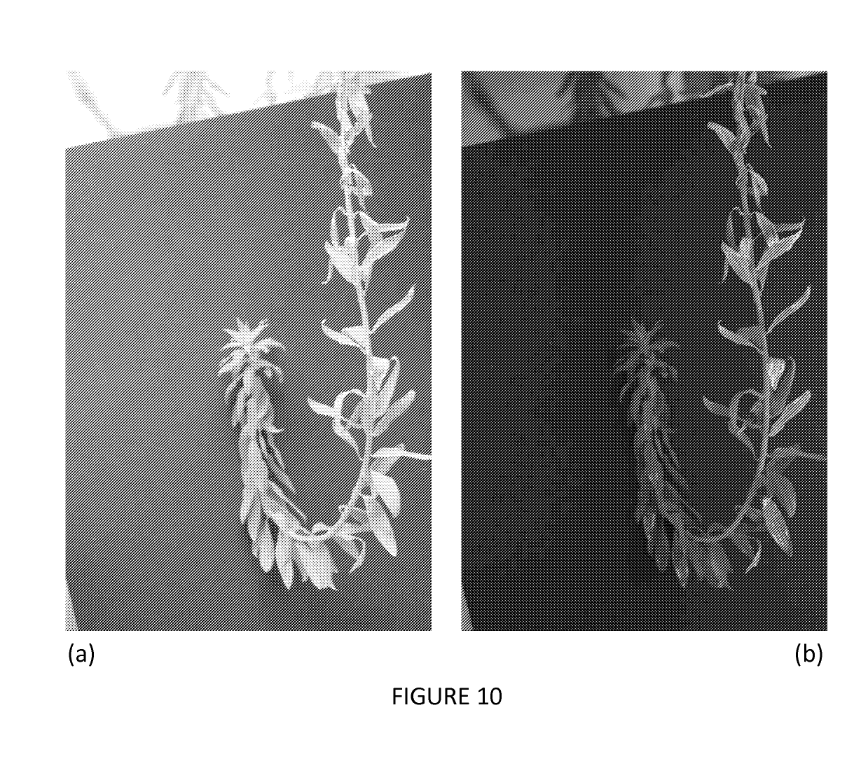

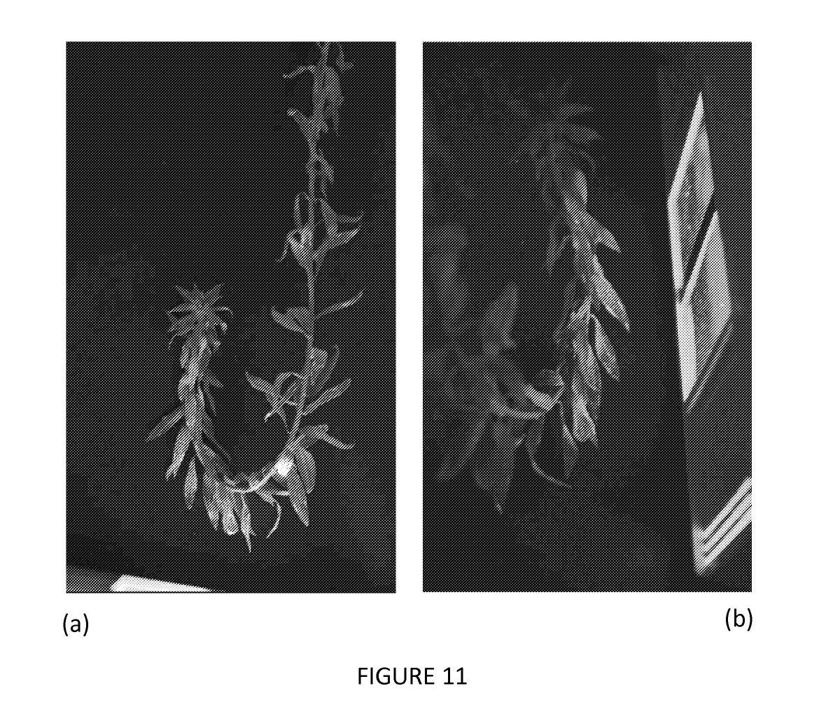

FIGS. 10 and 11 illustrate photographs of a flax plant according to a hydroponic composition described herein wherein the aqueous solution of the composition included D-282 dye. FIG. 10(a) is a photograph of the flax plant when exposed to ambient visible light. FIGS. 10(b), 11(a), and 11(b) are photographs of the flax plant when exposed to UV light. As illustrated in FIGS. 10(b), 11(a), and 11(b), the flax plant exhibited fluorescence under UV illumination.

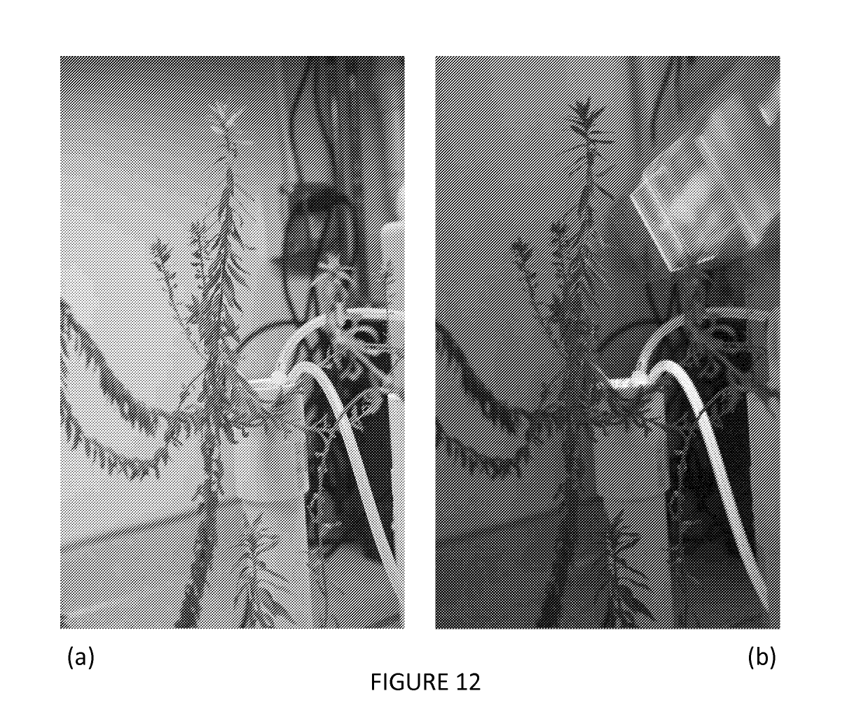

FIG. 12 illustrates photographs of a flax plant according to a hydroponic composition described herein wherein the aqueous solution of the composition included graphene nanoparticles. FIG. 12(a) is a photograph of the flax plant when exposed to ambient visible light. FIG. 12(b) is a photograph of the flax plant when exposed to UV light. As illustrated in FIG. 12(b), the flax plant exhibited fluorescence under UV illumination.

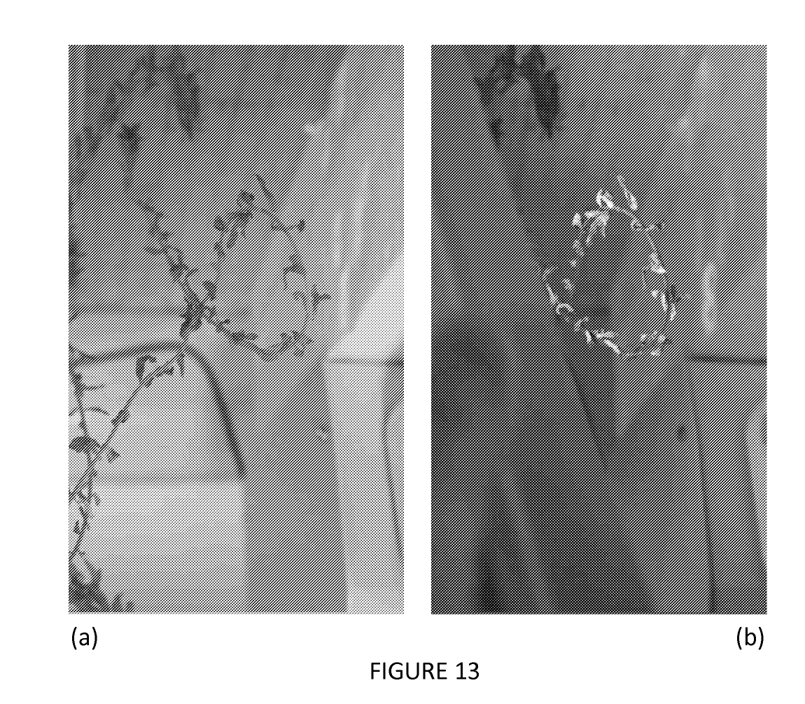

FIG. 13 illustrates photographs of a flax plant according to a hydroponic composition described herein wherein the aqueous solution of the composition included SWNTs. FIG. 13(a) is a photograph of the flax plant when exposed to ambient visible light. FIG. 13(b) is a photograph of the flax plant when exposed to UV light. As illustrated in FIG. 13(b), the flax plant exhibited fluorescence under UV illumination.

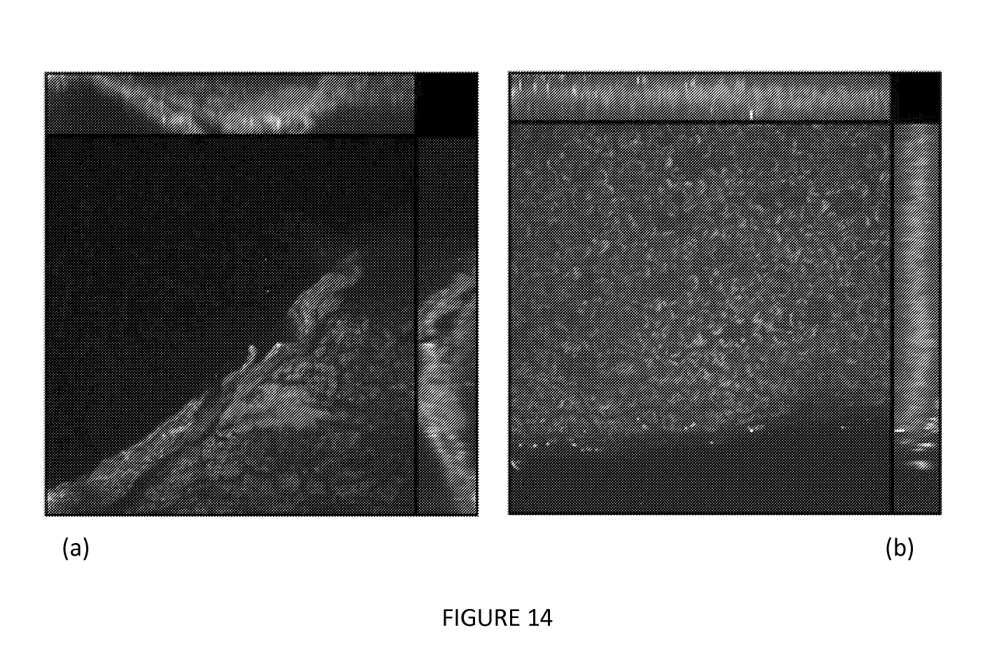

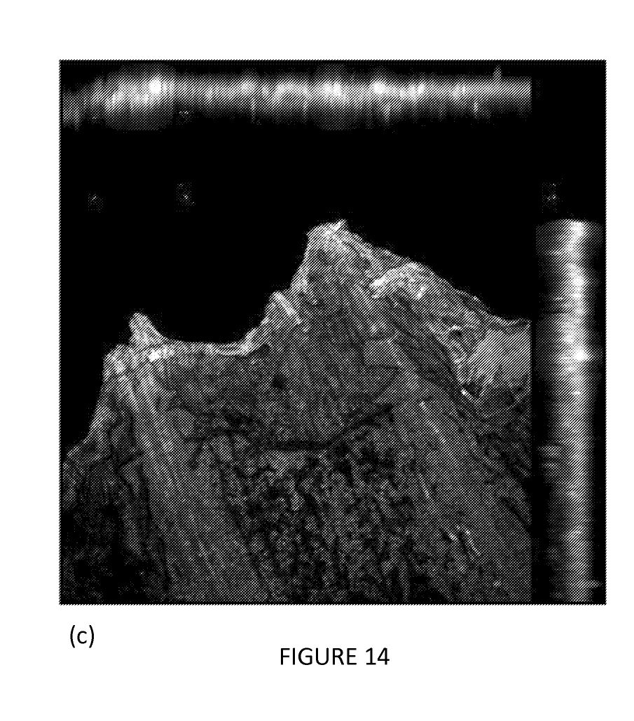

FIGS. 14(a)-(c) illustrate confocal microscopic Raman shift images of portions of a control flax plant and flax plants of hydroponic compositions described herein. Specifically, FIG. 14(a) illustrates a flax leaf tip including D-282 dye. FIG. 14(b) illustrates a flax leaf tip of a control flax plant. FIG. 14(c) illustrates a flax leaf tip including SWNTs. Irradiation was carried out at 405 nm, 488 nm, and 561 nm.

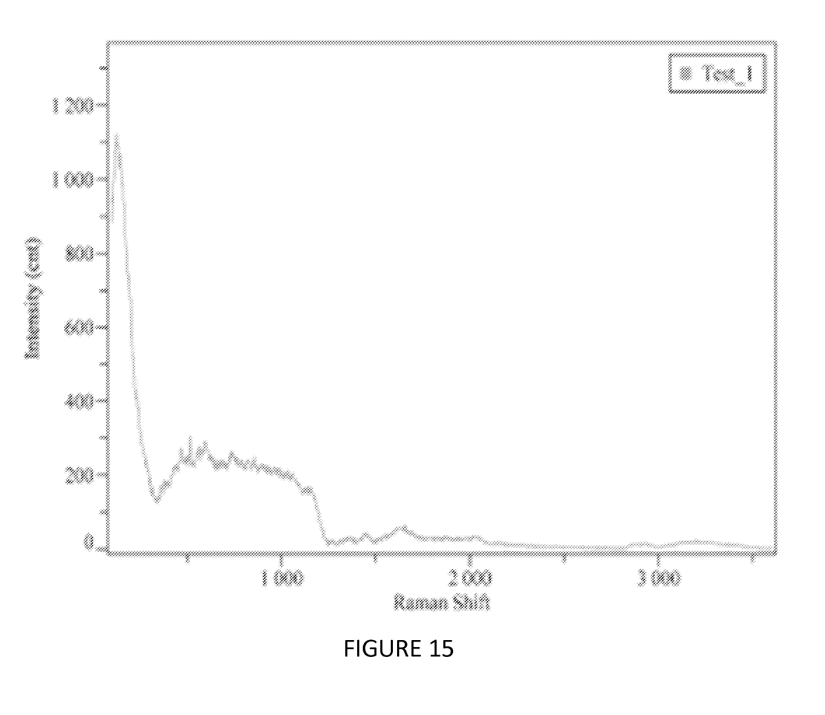

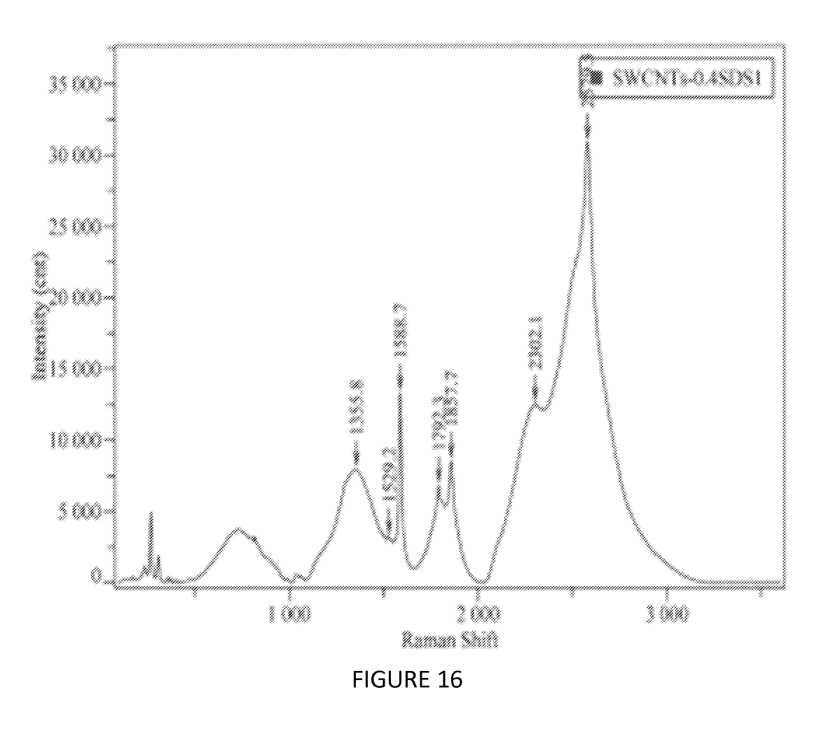

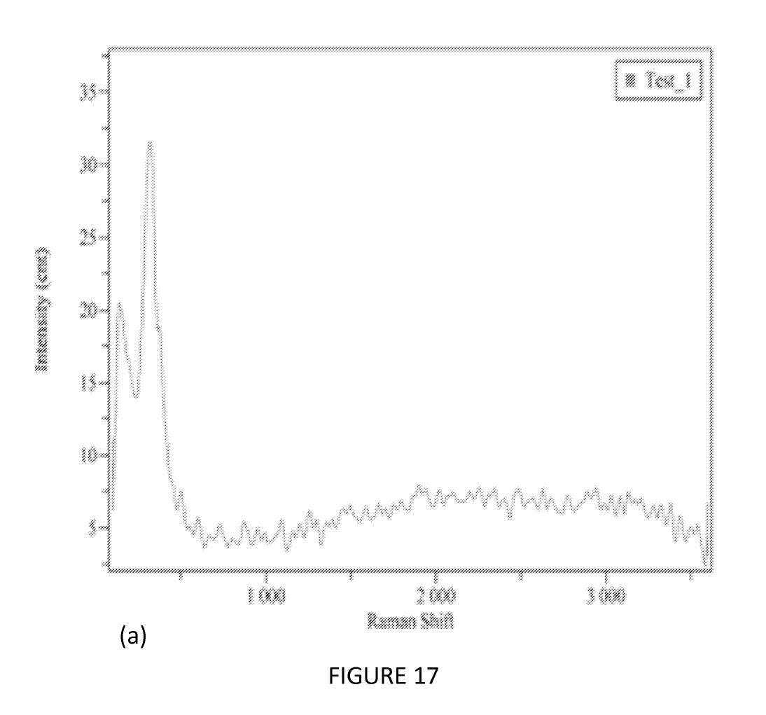

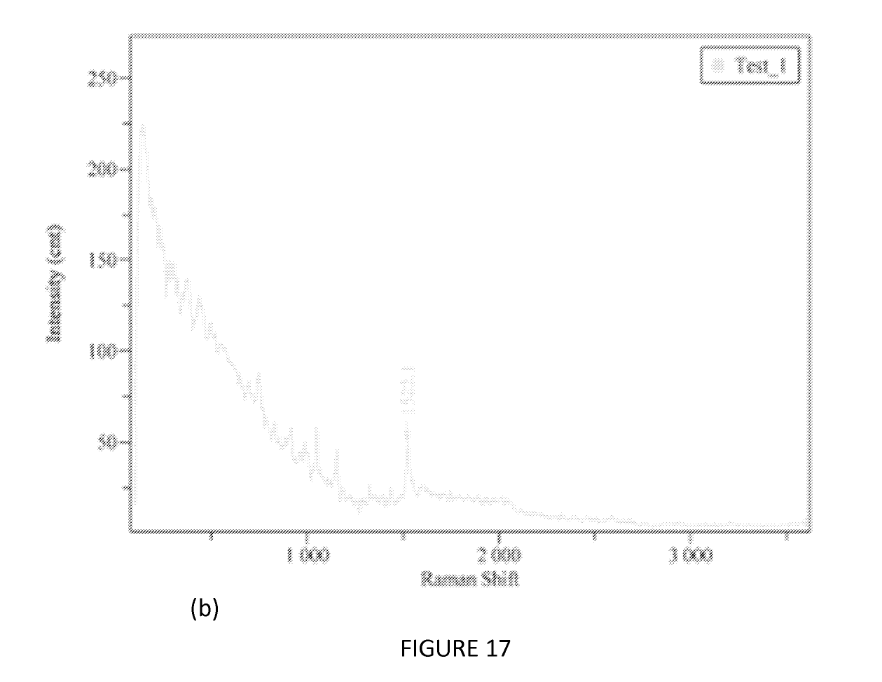

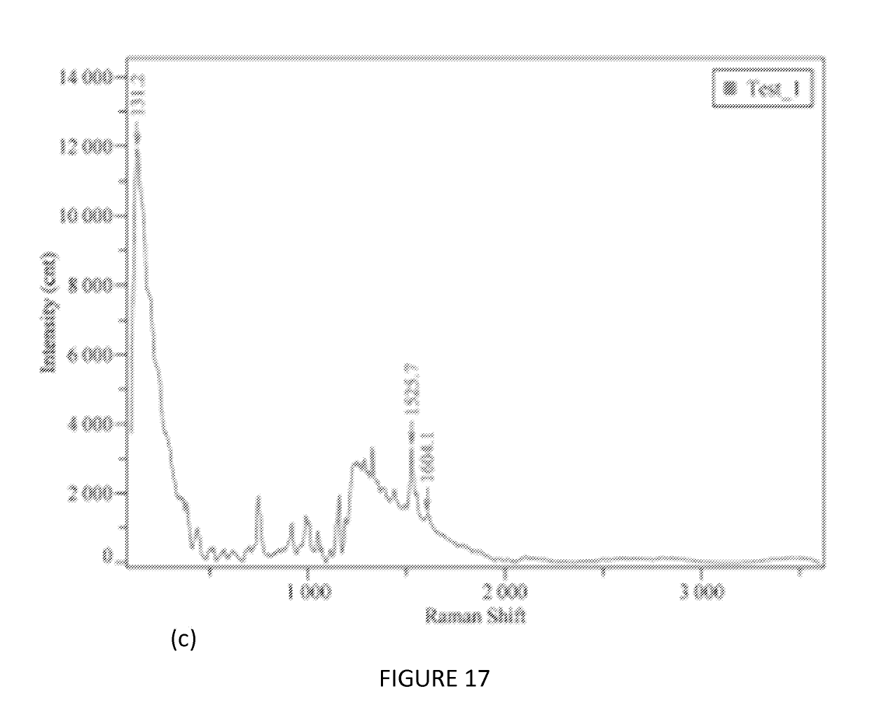

As controls for hydroponic compositions described herein, FIG. 15 illustrates a Raman spectrum of an SDS-SC surfactant solution, and FIG. 16 illustrates a Raman spectrum of SWNTs disposed in the surfactant solution of FIG. 15. Also as controls, FIGS. 17(a)-(c) illustrate Raman spectra of portions of a control flax plant stem. Specifically, the stem of the flax plant was cut in half in a direction perpendicular to the long axis of the stem (thus, the cut exposed a cross section of the stem). FIG. 17(a) corresponds to the surface of the top half of the stem. FIG. 17(b) corresponds to the top half of the stem following partial pulverization or chopping of the top half of the stem. FIG. 17(c) corresponds to the bottom half of the stem following partial pulverization or chopping of the bottom half of the stem. In all of FIGS. 15-17, NIR laser irradiation (785 nm) was used to obtain the spectra.

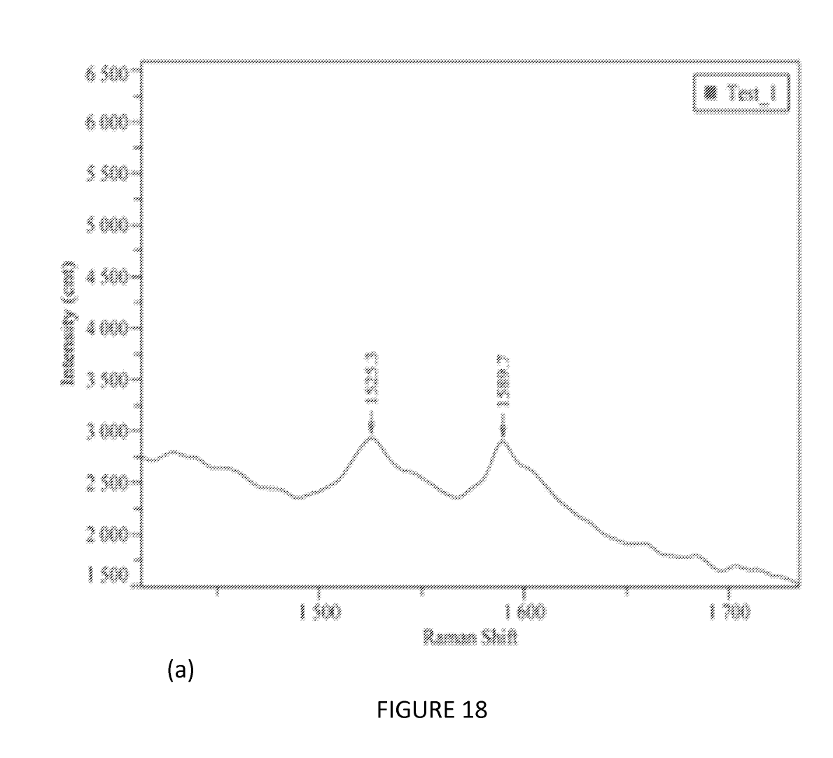

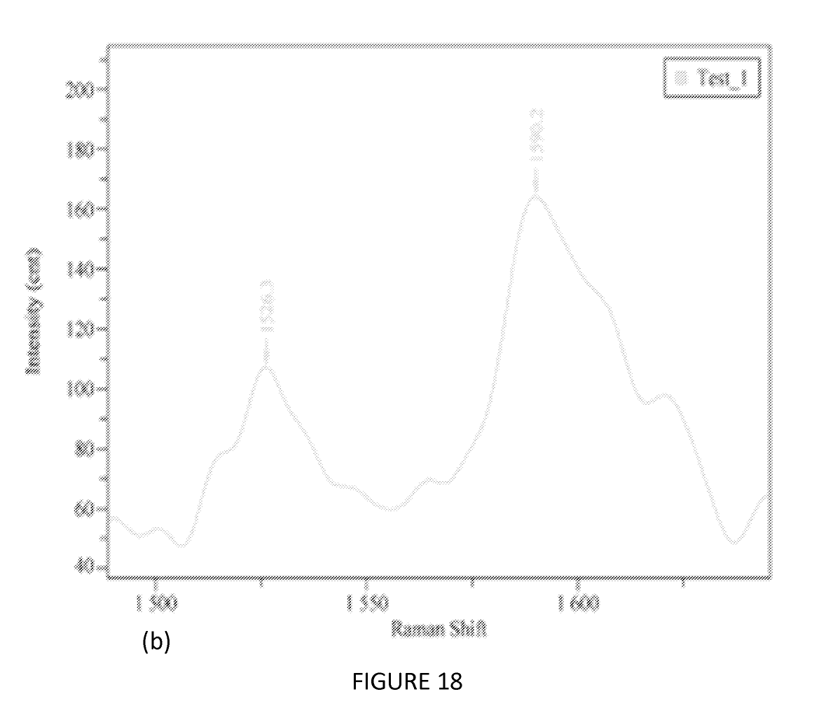

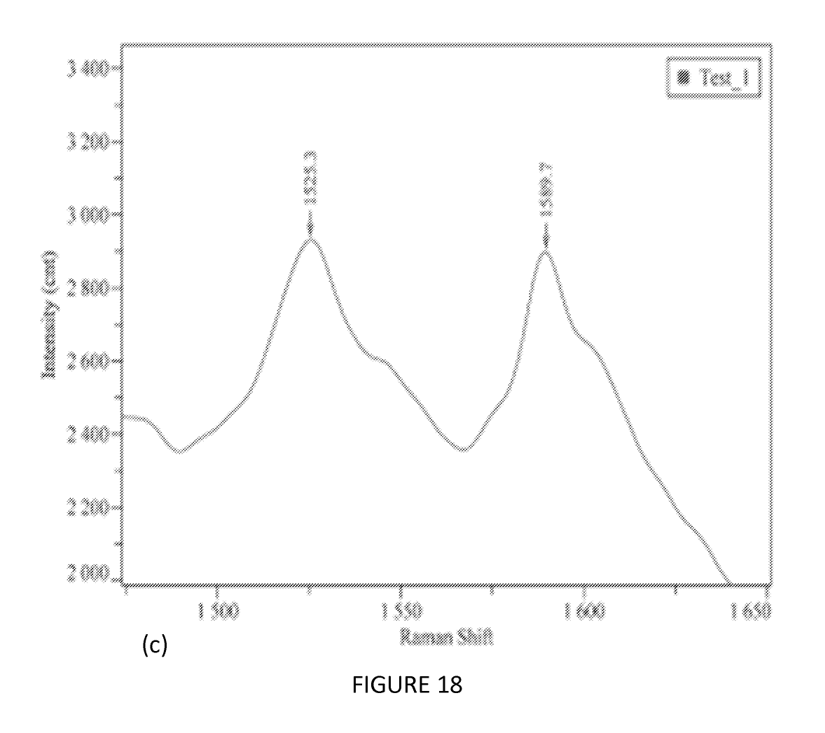

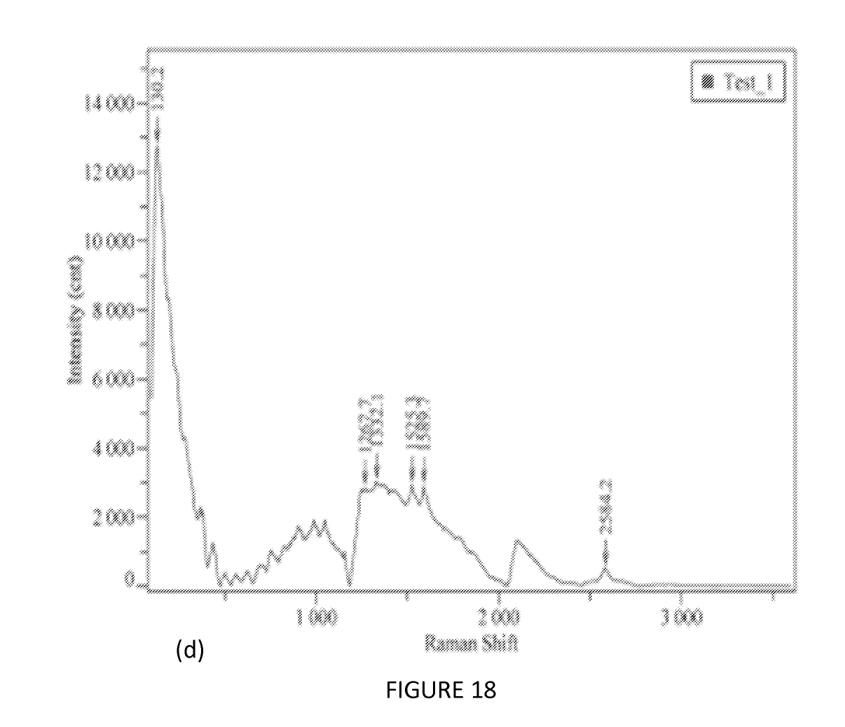

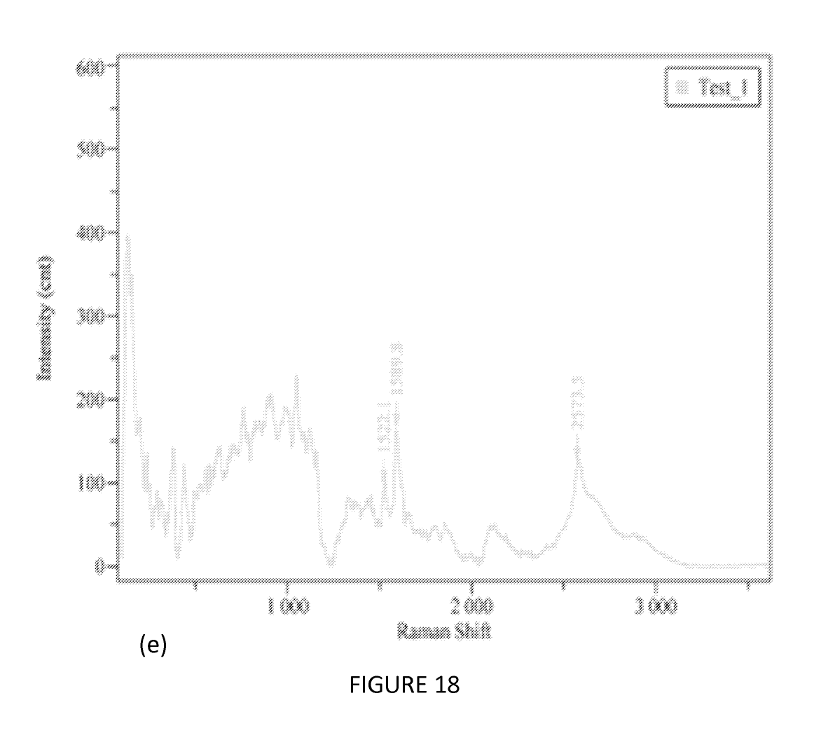

In contrast to FIGS. 17(a)-(c), FIGS. 18(a)-(e) illustrate Raman spectra (785 nm NIR laser irradiation) of portions of a flax plant stem of a hydroponic composition described herein wherein the aqueous solution of the composition included SWNTs disposed in SDS-SC. Following treatment with the aqueous solution, a slice was removed from the middle of the flax stem, resulting in a top half of the stem, a bottom half of the stem, and a middle slice of the stem. FIGS. 18(a), 18(c), and 18(d) correspond to the bottom half of the stem. FIGS. 18(b) and 18(e) correspond to the middle slice of the stem.

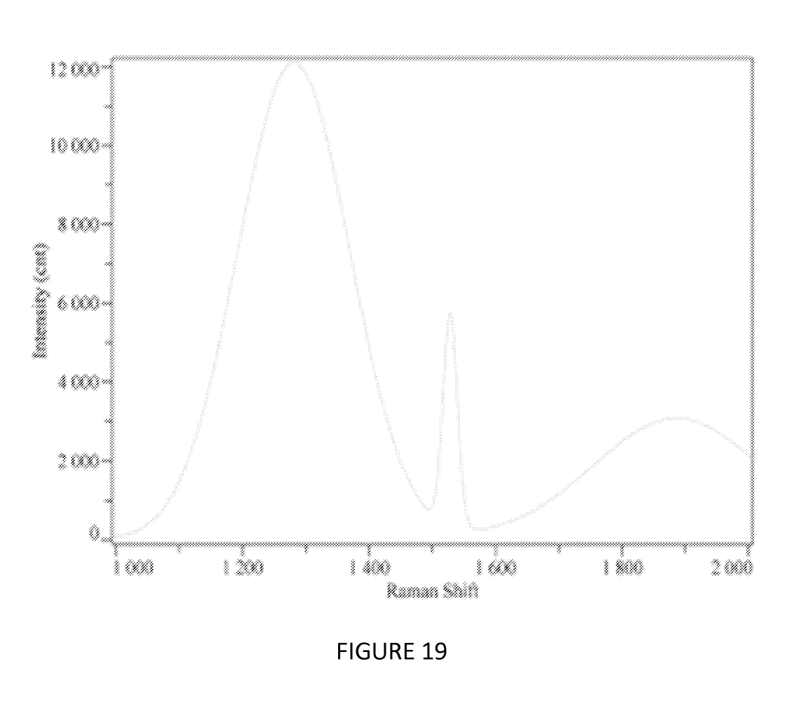

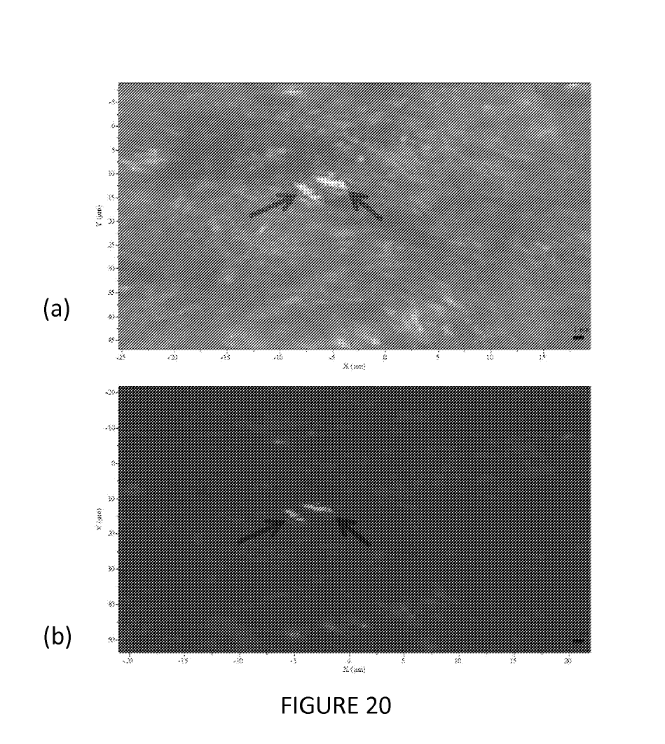

FIG. 19 illustrates a Raman spectrum (785 nm NIR laser irradiation) of a flax leaf tissue system of a hydroponic composition described herein wherein the aqueous solution of the composition included SWNTs disposed in SDS-SC. For comparison, a Raman spectrum of the leaf system of a control flax plant (not shown) did not exhibit a peak at 1530 cm⁻¹. In addition, FIGS. 20(a)-(b) illustrate Raman images of nanoparticles (marked with arrows) observed in flax leaves of the hydroponic composition. FIG. 20(a) corresponds to a visual image. FIG. 20(b) corresponds to a fluorescence image.

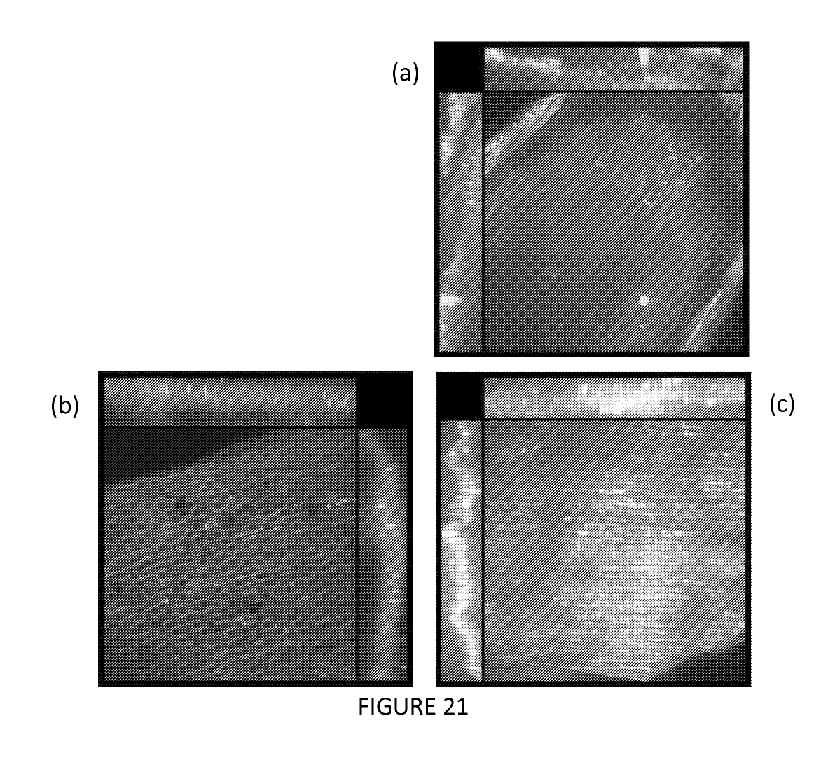

FIGS. 21(a)-(c) illustrate fluorescence images of portions of a control flax plant stem and the stem of a flax plant of a hydroponic composition described herein. Specifically, FIG. 21(a) corresponds to a fluorescence image of a sliced cross section of the stem of the control flax plant. FIG. 21(b) corresponds to the outer surface of the stem of a flax plant treated with an aqueous composition comprising SWNTs. FIG. 21(c) corresponds to a sliced cross section of the stem of the plant of FIG. 21(b).

Further results are provided in Table III below. Table III provides the distribution of various nanoparticles in different portions of a flax plant (stem or leaf) of a hydroponic composition described herein. In Table III, “Dry Mass” is the mass of the flax tissue when dried as described above. “V_{f} Solution” is the total volume of the aqueous solution provided to the plant; “ppm Metal V_{f}” is the amount of the relevant nanoparticle metal per mL of the aqueous solution; “ppm M total” is the total amount of the metal in the aqueous solution; and “Metal Mass” is the atomic mass of the relevant metal.

TABLE III
Nanoparticle Distributions in Flax

| | Flax | Dry | V_{f} | ppm | ppm | ppm M/g | Metal | | Molar | Particle |
|---|---|---|---|---|---|---|---|---|---|---|
| Nanoparticle | Tissue | Mass | Solution | Metal/ | M | dry | Mass | Molecular | Mass | uptake |
| Sample | Type | (g) | (mL) | V_{f} | Total | tissue | (g) | Formula | (g/mol) | (ppm) |
| Control | Stem | 0.410 | 3.900 | Noise | | | | | | |
| Control | Leaf | 0.580 | 4.900 | Noise | | | | | | |
| Alumina | Stem | 0.110 | 4.500 | Noise | | | | | | |
| Alumina | Leaf | 0.090 | 5.200 | 0.18 | 0.94 | 10.40 | 26.98 | Al₂O₃ | 101.96 | 39 |
| Silica | Stem | 0.160 | 5.000 | 0.767 | 3.84 | 23.97 | 28.09 | SiO₂ | 60.08 | 51 |
| Silica | Leaf | 0.150 | 4.800 | 2.639 | 12.67 | 84.45 | 28.09 | SiO₂ | 60.08 | 181 |
| ZnO | Stem | 0.380 | 4.700 | 8.864 | 41.66 | 109.63 | 65.41 | ZnO | 81.41 | 136 |
| ZnO | Leaf | 0.550 | 5.500 | 6.687 | 36.78 | 66.87 | 65.41 | ZnO | 81.41 | 83 |

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A hydroponic composition comprising:
an aqueous medium including one or more spectral additives; and
a plant in contact with the aqueous medium, wherein the one or more spectral additives are incorporated into a structure of the plant altering spectral properties of the plant.

2. The hydroponic composition of claim 1, wherein the spectral additives are luminescent species having absorption spectra non-overlapping with chlorophylls a and b.

3. The hydroponic composition of claim 2, wherein the luminescent species have emission spectra overlapping with the absorption spectra of chlorophylls a and b.

4. The hydroponic composition of claim 3, wherein the luminescent species downconvert UV radiation, near-UV radiation, or visible radiation for absorption by chlorophylls a and b.

5. The hydroponic composition of claim 3, wherein the luminescent species upconvert infrared radiation for absorption by chlorophylls a and b.

6. The hydroponic composition of claim 3, wherein the luminescent species absorb radiation in the 500-600 nm range.

7. The hydroponic composition of claim 1, wherein the spectral additives have absorption profiles substantially overlapping with that of chlorophylls a and b.

8. The hydroponic composition of claim 1, wherein the spectral additives reflect light in the visible region of the electromagnetic spectrum.

9. The hydroponic composition of claim 1, wherein the spectral additives comprise nanoparticles, laser dyes, anti-Stokes materials, anti-counterfeiting dye or a mixture thereof.

10. The hydroponic composition of claim 1, wherein the spectral additives comprise metal oxide nanoparticles.

11. The hydroponic composition of claim 1, wherein the spectral additives comprise carbon nanoparticles.

12. The hydroponic composition of claim 1, wherein the spectral additives are modified with a plant binding functionality.

13. The hydroponic composition of claim 12, wherein the plant binding functionality is a primary metabolite.

14-23. (canceled)

24. A method of altering the spectral properties of a plant comprising:
providing an aqueous medium including one or more spectral additives; and
contacting a plant with the aqueous medium to provide a hydroponic composition and initiate uptake of the one or more spectral additives by the plant, wherein the spectral additives are incorporated into the structure of the plant.

25. The method of claim 24, wherein the spectral additives are luminescent species having absorption spectra non-overlapping with chlorophylls a and b.

26. The method of claim 25, wherein the luminescent species have emission spectra overlapping with the absorption spectra of chlorophylls a and b.

27. The method of claim 26, wherein the luminescent species downconvert UV radiation or near-UV radiation for absorption by chlorophylls a and b.

28. The method of claim 26, wherein the luminescent species upconvert infrared radiation for absorption by chlorophylls a and b.

29-30. (canceled)

31. The method of claim 24, wherein the spectral additives reflect light in the visible region of the electromagnetic spectrum.

32. The method of claim 24, wherein the spectral additives comprise quantum dots, laser dyes, anti-Stokes materials, anti-counterfeiting dye, metal oxide nanoparticles or a mixture thereof.

33. (canceled) CHARLOTTE 142595. 1 5
